(19) United States
(12) Patent Application Publication
(10) Pub. No.: US 2021/0353210 A1
(43) Pub. Date: Nov. 18, 2021

(54) ELECTRIC DEVICE FOR THE SKIN ALLERGY TEST

(71) Applicant: Mediform Italia S.r.l., Acireale (CT) (IT)

(72) Inventor: Mario Mancini, Acireale (CT) (IT)

(21) Appl. No.: 17/278,299

(22) PCT Filed: Nov. 8, 2018
(86) PCT No.: PCT/IT2018/000145

§ 371 Date: Mar. 20, 2021

(51) Int. Cl. A61B 5/00; A61M 37/00

(52) U.S. Cl. CPC A61B 5/411; A61B 5/445; A61B 2560/04; A61M 2037/0007; A61M 37/00

(57) ABSTRACT

An electric device for performing a skin allergy test. The device has a main body having a metallic tip installed on the top thereof, and means of generation, transmission and control of an electric pulse contained inside the main body and electrically connected to the tip. The device includes a hollow outer nozzle having a cylindrical shape is located at the top of the main body that contains the tip inside so that it is not visible from outside. The device further includes means with a related tank releasing ink so that when the nozzle and the tip are placed in contact with a patient's skin then a series of ink marks are printed in sequence on the skin. Furthermore, an electric pulse is emitted on command, for each of the ink marks so that the pulse emitted from the tip generates a respective small surface hole on the skin.

The present invention regards an electric medical device, used for the skin allergy test, to be placed in touch with a skin, preferably a forearm skin belonging to a person. It is able to print a series of areas by ink marks, and to emit on command an electric pulse, that generates a respective small surface hole on the same skin.

The device comprises means providing on command one or more allergens, by drops or by liquid micro-film, to said areas printed by ink marks, so that the allergens could go in touch with the skin surface layers, and would be possible to observe the occurring of a possible allergy reaction.

As known, in the state of the art there is the so called prick test, that represents a skin allergy test, and it is very important for the diagnosis of the respiratory diseases, nutritional diseases, and diseases caused by other allergens. The prick test is an allergy test used in order to understand when a specific substance causes an allergic inflammation, by immediate mechanism or by an IgE (Immunoglobulin E) mechanism, that is an immediate-type hypersensitivity or a reaction of the first type, according to the Coombs and Gell classification.

In the prick test, some drops of purified allergen are placed on the skin surface, usually on the forearm, and then the skin is scratched by a single-use lancet, in order to make the allergen to penetrate under the skin surface layers. After waiting approximately 15-20 minutes, the skin reaction is observed, with reference to each specific allergen that has been applied. The reaction to each single allergen is compared with a positive check (usually by using histamine) and with a negative check (usually by using glycerin, that is the standard dilution liquid for the allergens).

In case of a positive reaction, it is possible to observe a so called hive, that is a skin edema, similar to a mosquito bite, that is very itchy and has a perimetric erythema ring.

However, this simple test is characterized by a drawback connected to the standardization of the procedure, because it is quite impossible to repeat the same depth in penetration by different lancets. In fact, it is quite impossible to repeat exactly the same pressure and location of the lancet on the skin.

Another drawback is related to the location of drops after each application at one point, with a consequent diffusion and contamination of the adjacent application sites.

Furthermore, this type of technique leads to an effect, so called belonephobia (otherwise known as trypanophobia), that could cause even the state of unconsciousness in a patient affected by panic.

All the above drawbacks are overcome by the present invention, that discloses a medical device of new generation, that guarantees a perfect standardization in the procedure of the skin allergy test, due to the emission of an electric pulse having low voltage and high amperage (ion-carbonation)—technical reference: battery 850 mAh 3.7 V, input current 5V/1 A, max output power 5 W.

Furthermore, the same device permits to decrease the execution time of the procedure, without pain or bleeding for the patient.

Therefore, the main objective of the present invention to propose an electric device for the skin allergy test, to be placed in touch with a skin, preferably a forearm skin belonging to a person; said device being able to print a series of areas by ink marks, and to emit on command an electric pulse, that generates a respective small surface hole on the same skin.

Another objective is that said device comprises means providing on command one or more allergens, by drops—e.g. by a pipette or a brush dispenser, to said areas printed by ink marks, so that the allergens could go in touch with the skin surface layers, and would be possible to observe the occurring of a possible allergy reaction.

A further objective is that intensity and duration of the electric pulse emitted by the device could be defined by: tuning means based on a manual command; or tuning means based on an automatic command (skin auto-checking); or means that are set to factory default values.

A further objective is that the device comprises an add-on element, in a set back or front position, and that said add-on element comprises a set of elements placed on a platform.

And another further objective is that the same device comprises, at the external surface, a series of buttons for activation and/or selection of the pulse duration and intensity, and it further comprises some LED elements, having green or red color, that indicate different states of working in the same device.

Therefore, it is specific subject of the present invention an electric device for the skin allergy test, characterized in that comprising:

a main body, where a metallic tip is installed on the top of it;

means of generation, transmission and control of an electric pulse, said means being contained inside said main body, and being electrically connected to the same tip;

an outer nozzle, that is made of plastic, having a cylindrical and hollow shape, placed at the top of said main body, that contains said tip inside, so that it is not visible from outside; and including further means with a related tank releasing ink, so that said nozzle and said tip are placed in touch with a skin, preferably a forearm skin belonging to a person; a series of ink marks, that are shaped as circular areas, are printed in sequence on the same skin; an electric pulse is emitted on command, for each of said circular areas, so that the same pulse emitted from said tip generates a respective small surface hole on the same skin.

The present invention is now being described according to non-limiting examples, with particular reference to the figures of the enclosed drawings, where:

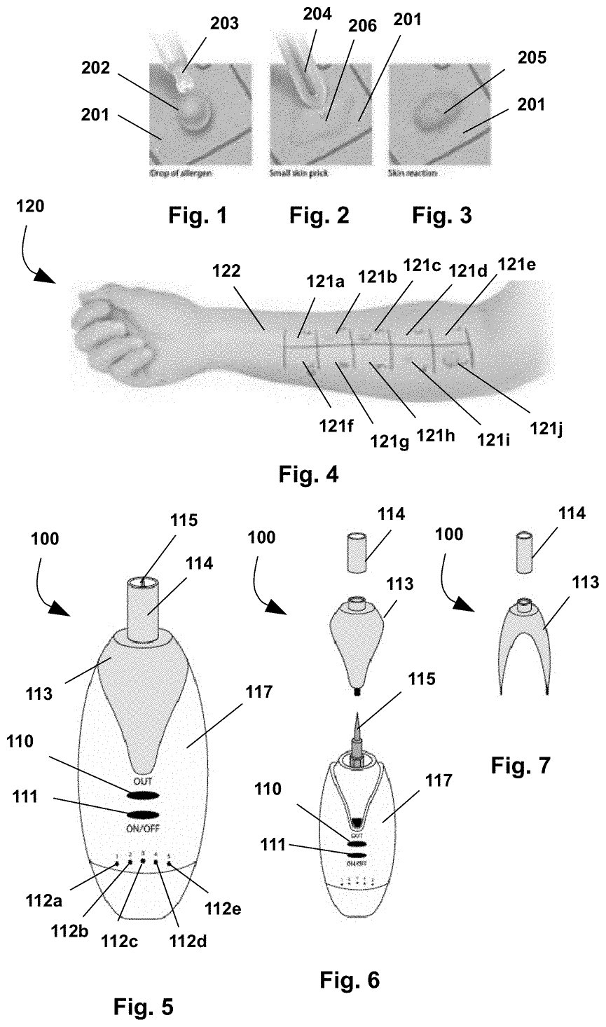

FIG. 1 is a perspective view where an allergen drop is placed on the surface skin belonging to a person;

FIG. 2 is a perspective view where a single-use lancet scratches the same skin of FIG. 1, so that the allergen liquid can penetrate into the skin surface;

FIG. 3 is a perspective view where the same skin of FIG. 1 has a positive reaction to the same allergen, and a skin edema shows up, called hive, having a perimetric erythema ring;

FIG. 4 is a front perspective view of the forearm belonging to a person, where a series of areas have been ink marked, and on each of them an allergy test has been done;

FIG. 5 is a front perspective view of an allergy test device, according to the present invention, in an assembled configuration;

FIG. 6 is a front perspective view of the same allergy test device of FIG. 5, in a disassembled configuration;

FIG. 7 is a front perspective view of components belonging to the same allergy test device, in a disassembled configuration, which are a nozzle and an ink tank;

FIG. 8 is a front perspective view of a prototype representing the same allergy test device;

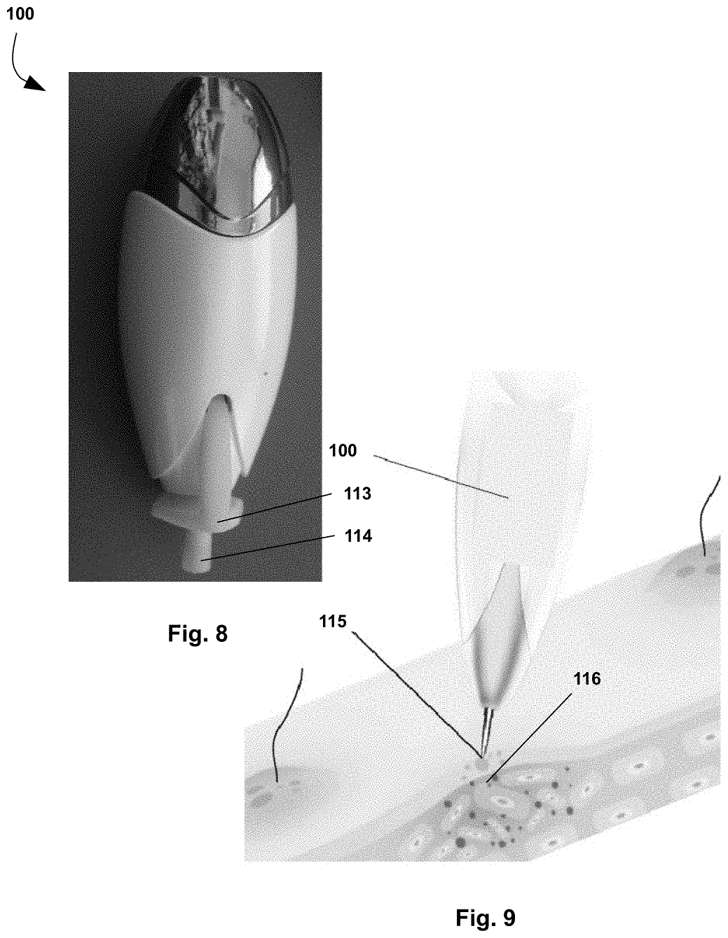

FIG. 9 is a front schematic perspective view of the same allergy test device applied to a skin surface;

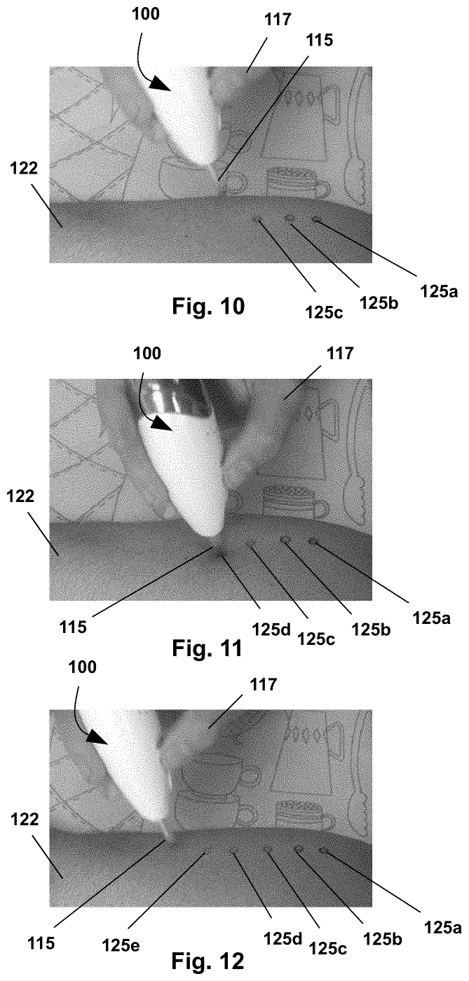

FIGS. 10-12 are respective front views of a prototype representing the same allergy test device, in a sequence of steps applying to a skin surface;

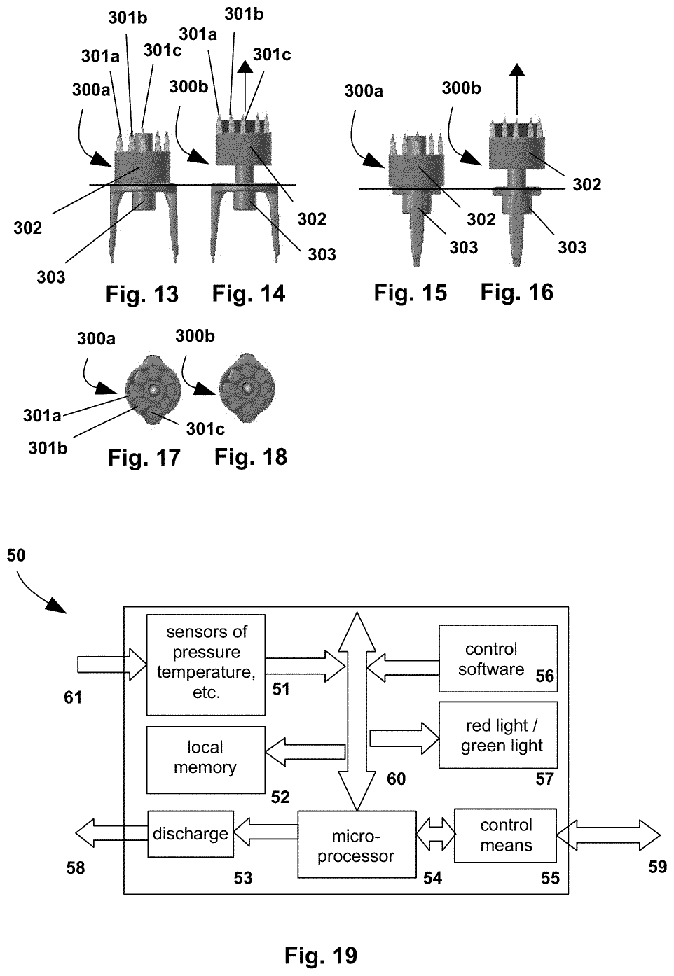

FIGS. 13, 15 and 17 are respective front, lateral and top views, of an add-on element in a set back position, applied to the same allergy test device;

FIGS. 14, 16 and 18 are respective front, lateral and top views, of an add-on element in a set front position, applied to the same allergy test device;

FIG. 19 is a view of a block diagram related to means of generation, transmission and control of an electric pulse, belonging to the same allergy test device.

It is underlined that only few of the many conceivable embodiments of the present invention are here described, which are just some specific non-limiting examples, having the possibility to describe many other embodiments based on the disclosed technical solutions of the present invention.

FIGS. 5, 6, 7 and 8 show an electric device 100 for the skin allergy test, essentially comprising: a main body 117, where a metallic tip 115 is installed on the top of it.

FIG. 9 shows more in detail how tip 115 is placed in touch with a skin 116, preferably a forearm 122 skin belonging to a person, as that shown in FIG. 4.

In order to work, the same electric device 100 further comprises means 50 of generation, transmission and control of an electric pulse. Said means 50 are contained inside said main body 117, and are electrically connected to the same tip 115.

The electric device 100 further comprises an outer nozzle 114, that is made of plastic, having a cylindrical and hollow shape, placed at the top of said main body 117, that contains said tip 115 inside, so that it is not visible from outside; and including further means with a related tank releasing ink 113.

With reference to FIGS. 9, 10, 11 and 12, it is possible to see a sequence of steps related to the use of the device: said nozzle 114 and said tip 115 are placed in touch with a skin 116, preferably a forearm skin belonging to a person; a series of ink marks, that are shaped as circular areas 125a, 125b, . . . , are printed in sequence on the same skin 116; an electric pulse is emitted on command, for each of said circular areas 125a, 125b, . . . , so that the same pulse emitted from said tip 115 generates a respective small surface hole on the same skin 116.

Further according to the invention, the device 100 comprises means for storage of respective substances, by drops or by liquid micro-film, each of said substances being composed of a specific purified allergen.

The tip 301a, 301b, . . . , of each allergen dispenser is shaped as a small brush, and when it is passed on the skin it releases an allergen micro-film that is smaller than a drop, avoiding therefore the drawback of liquid diffusion and contamination with other points. With reference to FIGS. 1, 2 and 3, there are shown means providing on command 203 each of said substances, by drops 202—e.g. by a pipette or a brush dispenser, from said means for storage to a respective area 201 of said circular areas 125a, 125b, . . . .

In such a way, the drops 202 of substances go in touch with the respective skin 116 surface layers, and it is possible to observe, after a specific time, the occurring of a possible allergy reaction with reference to each of said substances: in case of a positive allergy reaction a so called hive 205 shows up, that is a skin edema having a perimetric erythema ring.

FIG. 19 shows a block diagram of said means 50 of generation, transmission and control of an electric pulse. They are composed of: a micro-processing unit 54; a data bus 60; a local memory 52; a control software 56; external control means 55, e.g. represented by buttons; some LED elements 57, e.g. represented by red light/green light; sensors of external physical magnitudes 51, e.g. represented by pressure, temperature, conductivity, etc.; and a generator of an electric discharge 53.

Said means 50 of generation, transmission and control of an electric pulse, can further comprise means tuning on manual command of intensity and duration of the emitted pulse, corresponding to one of the following levels: for more sensitive skins, for less sensitive skins, and for resistant skins (old people).

As an alternative, said means 50 of generation, transmission and control of an electric pulse, can further comprise: sensors for detection of the skin resistance, and means tuning on automatic command (skin auto-checking) of intensity and duration of the emitted pulse, corresponding to the type of detected skin.

Said means 50 of generation, transmission and control of an electric pulse, can further comprise means defining the intensity and duration of the emitted pulse according to factory default values.

FIGS. 13, 15 and 17 show an add-on element 300a, in a set back position, applied to the same allergy test device 100. Said add-on element 300a comprises a set of elements 301a, 301b, 301c, . . . , placed on a platform 302 connected to a supporting element 303.

FIGS. 14, 16 and 18 show an add-on element 300b, in a set front position, applied to the same allergy test device 100. Said add-on element 300b comprises a set of elements 301a, 301b, 301c, . . . , placed on a platform 302 connected to a supporting element 303.

The present invention further discloses a process to achieve a skin allergy test, by using an electric device 100, as defined according to technical characteristics above described. The process comprises the following steps:

said nozzle 114 and said tip 115 are placed in touch with a skin 116, preferably a forearm skin belonging to a person;

a series of ink marks, that are shaped as circular areas 125a, 125b, . . . , are printed in sequence on the same skin 116;

an electric pulse is emitted on command, for each of said circular areas 125a, 125b, . . . , so that the same pulse emitted from said tip 115 generates a small surface hole on the same skin 116;

providing on command 203 a specific purified allergen, by drops 202, to a respective area 201 of said circular areas 125a, 125b, . . . , so that the drops 202 go in touch with the respective skin 116 surface layers;

observing, after a specific time, the occurring of a possible allergy reaction with reference to each of said allergen substances; in case of a positive allergy reaction a so called hive 205 shows up, that is a skin edema having a perimetric erythema ring.

In the above process to achieve a skin allergy test, the intensity and duration of the electric pulse can be defined by: tuning means based on a manual command; or tuning means based on an automatic command (skin auto-checking); or means that are set to factory default values.

Therefore, the above examples show that the present invention reaches all the expected objectives. In particular, it permits to achieve electric device for the skin allergy test, to be placed in touch with a skin, preferably a forearm skin belonging to a person; said device is able to print a series of areas by ink marks, and to emit on command an electric pulse, that generates a respective small surface hole on the same skin.

Furthermore, the device comprises means providing on command one or more allergens, by drops—e.g. by a pipette or a brush dispenser, to said areas printed by ink marks, so that the allergens can go in touch with the skin surface layers, and it is possible to observe the occurring of a possible allergy reaction.

Further according to the invention, the intensity and duration of the electric pulse emitted by the device can be defined by: tuning means based on a manual command; or tuning means based on an automatic command (skin auto-checking); or means that are set to factory default values.

The invention further comprises an add-on element, in a set back or front position, and that said add-on element comprises a set of elements placed on a platform.

Finally, according to the invention, the same device comprises, at the external surface, a series of buttons for activation and/or selection of the pulse duration and intensity, and it further comprises some LED elements, having green or red color, that indicate different states of working in the same device.

The present invention has been described by making reference to some non-limiting examples and following some preferred embodiments; however it goes without saying that modifications and/or changes could be introduced by those skilled in the art without departing from the relevant scope, as defined in the enclosed claims.

1. An electric device for performing a skin allergy test, the electric device comprising:
a main body, where a metallic tip is installed on the top of it;
means of generation, transmission and control of an electric pulse, said means being contained inside said main body, and being electrically connected to the same tip;
an outer nozzle, that is made of plastic, having a cylindrical and hollow shape, placed at the top of said main body, that contains said tip inside, so that it is not visible from outside; and including further means with a related tank releasing ink, so that said nozzle and said tip are configured to be placed in touch with a skin belonging to a person; a series of ink marks, that are shaped as circular areas, are printed in sequence on the same skin an electric pulse is emitted on command, for each of said circular areas, so that the same pulse emitted from said tip generates a respective small surface hole on the same skin.

2. The electric device according to claim 1, comprising:
means for storage of respective substances, each of said substances being composed of a specific purified allergen; and means providing on command each of said substances, by drops from said means for storage to a respective area of said circular areas, so that the drops of substances go in touch with the respective skin surface layers, and it is possible to observe, after a specific time, the occurring of a possible allergy reaction with reference to each of said substances: in case of a positive allergy reaction a so called hive shows up, that is a skin edema having a perimetric erythema ring.

3. The electric device according to claim 1 including:
said means of generation, transmission and control of an electric pulse, further comprise means tuning on manual command of intensity and duration of the emitted pulse, corresponding to one of the following levels: for more sensitive skins, for less sensitive skins, and for resistant skins.

4. The electric device according to claim 1 including:
said means of generation, transmission and control of an electric pulse, further comprise: sensors for detection of the skin resistance, and means tuning on automatic command (skin auto-checking) of intensity and duration of the emitted pulse, corresponding to the type of detected skin.

5. The electric device according to claim 1 wherein:
said means of generation, transmission and control of an electric pulse, further comprise means defining the intensity and duration of the emitted pulse according to factory default values.

6. The electric device according to claim 1 wherein:
said means of generation, transmission and control of an electric pulse, are composed of: a micro-processing unit; a data bus; a local memory; a control software; external control means; some LED elements; sensors of external physical magnitudes; and a generator of an electric discharge.

7. The electric device according to claim 1, wherein:
an add-on element, in a set back position, applied to the same allergy test device; said add-on element comprises a set of elements, placed on a platform connected to a supporting element.

8. The electric device according to claim 1, wherein:
an add-on element, in a set front position, applied to the same allergy test device; said add-on element comprises a set of elements, placed on a platform connected to a supporting element.
